(19) United States
(12) Patent Application Publication
(10) Pub. No.: US 2010/0144016 A1
(43) Pub. Date: Jun. 10, 2010

(54) APPARATUS FOR THE DISRUPTION OF ANIMAL CELLS

(75) Inventor: Sohail Rashid CHOHAN, Beckenham (GB)

Correspondence Address: GLAXOSMITHKLINE;Corporate Intellectual Property - UW2220, P.O. Box 1539, King of Prussia, PA, 19406-0939, US

(73) Assignee: Glaxo Group Limited

(21) Appl. No.: 12/625,813

(22) Filed: Nov. 25, 2009

Related U.S. Application Data
(63) Continuation of application No. 11/569,211, filed on Nov. 16, 2006, now abandoned.

(30) Foreign Application Priority Data
May 18, 2004 (GB) GB0411081.3

(51) Int. Cl. C12N 7/02; C12M 1/33

(52) U.S. Cl. 435/239; 435/306.1

(57) ABSTRACT

The present invention relates to the field of purifying recombinant virus propagated in animal cells. More particularly, it relates to a method for extracting virus from virus-infected cells that have been grown in culture in order to release virus and to apparatus for extracting virus from virus-infected cultured cells using the methods as described herein.

### CROSS-REFERENCE TO RELATED APPLICATION

The present application is a continuation of U.S. application Ser. No. 11/569,211, filed Nov. 16, 2006, which application is a §371 national phase entry of International Application No. PCT/GB2005/001867, filed May 16, 2005, which claims priority to International Application GB0411081.3, filed May 18, 2004, each of which is hereby incorporated by reference in its entirety.

### FIELD OF THE INVENTION

The present invention relates to the field of purifying recombinant virus propagated in animal cells. More particularly, it relates to a method for extracting virus from virus-infected cells that have been grown in culture in order to release virus and to apparatus for extracting virus from virus-infected cultured cells using the methods as described herein.

### BACKGROUND

Many biotechnological and fermentation processes require large amounts of cells to be grown and then disrupted to release desirable products. Cell disruption can be accomplished by mechanical, chemical, biological or physical means. It is highly desirable to eliminate the need for additional reagents such as detergents and also to avoid difficult physical methods such as freeze/thaw.

A number of mechanical methods have been developed to disrupt microorganisms. However, animal cells, which are becoming a common host cell of choice have a fragile membrane and thus require a more gentle disruption process to release the desirable products.

Examples of suitable viral vectors which may be harvested from animal cells include herpes simplex viral vectors, vaccinia or alpha-virus vectors and retroviruses, including lentiviruses, adenoviruses and adeno-associated viruses. Gene transfer techniques using these viruses are known to those skilled in the art. Retrovirus vectors for example may be used to stably integrate the polynucleotide or gene construct of interest into the host genome, although such recombination is not preferred. Replication-defective adenovirus vectors by contrast remain episomal and therefore allow transient expression.

One particular class of virus is the adenovirus. Recombinant adenoviruses are a class of viruses currently being used as gene therapy vectors to deliver therapeutics genes of interest to humans. Propagation of replication deficient adenoviruses are undertaken in mammalian cell lines such as the commercially available cell lines PER.C6® (CRUCELL), HEK293 (ATCC) or any other compatible virus packaging/complementing cell lines. In general, cells infected with adenovirus will eventually lyse to release the virus progeny (referred to as adenovirus mediated cell lysis). However from a processing view this stage is not preferable, as it will expose the virus to released proteolytic enzymes for an excessive period of time, which may degrade and inactivate the virus. Thus it is more preferable to harvest the virus from infected cells prior to adenovirus mediated cell lysis both in terms of maintaining greater product (virus) integrity and reducing the post infection harvest time.

Methods that are typically used for releasing adenovirus from infected cells for the purification of adenoviruses include:
a) freeze-thaw. This involves freezing the cell-containing culture and then thawing to disrupt the membrane. This must be done repeatedly at least three times. It is thus a time consuming process and only applicable to very small volumes.
b) detergent lysis. Detergents such as Tween 80®, Tween 20®, Triton X-100® or deoxycholate can be added to the cell culture to solubilise the plasma and nuclear membrane and release the virus.
c) mechanical lysis. Commercially available homogenisers or microfluiders operate at high pressures (>1000 psi) and under high shear. Homogenisation is the major mechanical method for release of product from microbial or animal cells. The typical. pressure ranges for microbial cell disruption are 5000 psi to 20,000 psi (see ‘protein purification, design and scale-up of downstream processing’, S. M. Wheelwright page 64, Wiley-Interscience). The amount of disruption that can be obtained at a given pressure depends on the type of organism/cell being disrupted. Organisms with stronger cell walls will require higher pressure to achieve a given level of breakage. Such high disruption pressures would not be necessary for lysis of viral-infected cells. Furthermore the high shear environment generated by a homogeniser could lead to inactivation of the viral product.

Shear force generated by adjustment of the transmembrane pressure on a tangential flow microfiltration has been used to disrupt mammalian cells to harvest adenovirus (see conf proc Williamsburg Nov. 16-19, 1998 ‘viral vector & vaccines’, Nancy Connelly et al.). Observations on the shear damage to different animal cells in a concentric cylinder viscometer have been documented, (Mardikar S H. et al., Biotech & Bioeng, Vol. 68, No. 6, Jun. 20, 2000). In this case it stated that a minimum shear rate is needed for disruption of cells. Cultured cells can also be disrupted using an impinging jet device (see U.S. Pat. No. 5,721,120).

It would be desirable to have an apparatus which efficiently disrupts animal cells and releases the cell content without damage.

### SUMMARY OF THE INVENTION

The present invention provides a method and apparatus for disrupting virus infected animal cells in a controlled manner which does not require high pressures and does not generate high inactivating shear forces for the recovery of virus from infected cells. Accordingly the present invention provides a method of extracting a virus from an animal cell infected with the virus, comprising the steps of;
a) providing cells in a suspending fluid,
b) pressurising the suspension of cells to a pressure of from 5 psi to 1000 psi, and
c) causing the pressurised suspension to flow through an restriction having a maximum diameter of 0.5 mm at a flow rate so that the cell membrane is ruptured.

In one embodiment the restriction is a valve. This valve could be a needle valve or ball valve (both of which are available commercially, for example Saunders or Swagelock). The valve may have a orifice diameter of up to 0.5 mm. For example from 0.05 mm to 0.2 mm, or from 0.1 mm to 0.2 mm.

In a further embodiment the restriction is a conduit such as a capillary device.

In particular the invention provides a method of extracting a virus from an animal cell infected with the virus, comprising the steps of;
a) providing cells in a suspending fluid,
b) pressurising the suspension of cells, and
c) causing the pressurised suspension to flow through a conduit having a maximum diameter of 0.5 mm at a flow rate so that the residence time of the cells in the conduit such that the cell membrane is ruptured. The flow rate can be varied by changing the pressure of the suspension, or by changing the bore size of the conduit, or by changing the cell density of the suspension.

The skilled man will be aware that certain parameters of the system i.e. the bore size and the length of the conduit, the pressure applied to the cell suspension and the cell density of the suspension, can be varied to create optimal conditions in which to lyse the cells. However if the parameters are varied too much then the levels of shear created within the conduit may become too high, causing product inactivation due to excessive foaming or over-exposure to regions of high shear.

The conduit may have a bore size of up to 0.5 mm. For example from 0.05 mm to 0.2 mm, or from 0.1 mm to 0.2 mm, in particular 0.13 mm. Bore dimensions can be varied so that sufficient shear force and friction force is created within the conduit. Furthermore, the conduit may have a length of between 0.5 cm-10 cm. For example from 2 cm to 8 cm, or from 4 cm to 6 cm. In particular it may have a length of 5 cm. The length of the conduit can be varied to generate the shear forces and sufficient residence time in the conduit to enable efficient disruption. Disruption of the cells results from the exposure of the cells to a shear field present in the conduit as a result of the driving pressure of the flow velocity (flow rate). Hence the flow rate can be varied, for example, by varying the pressure of the cell suspension, or by varying the dimensions of the bore. The length of the tube defines the residence time the cells spend in the shear field. Furthermore, the diameter of the conduit defines the distribution of the shear field across the cross-sectional area of the conduit. This is the area where cell disruption is caused by energy dissipation (kinetic energy) applied to the cells entering a high shear field.

Pumps suitable for the apparatus of the present invention cause low-shear and include peristaltic pumps, rotary lobe pumps (e.g. Quattro pump from Lenze, Germany), HPLC pumps, reciprocating pumps and diaphragm pumps. Alternatively the pressure could be provided by gases such as nitrogen and compressed air or an air compressor of suitable capacity (such as the JUN-AIR, Denmark).

In one embodiment the pressure provided by the pump is from 5 psi to 1000 psi. For example from 10 to 250 psi, from 25 to 250 psi, from 10 to 100 psi, from 25 psi to 60 psi, from 15 to 70 psi, or from 30 to 50 psi. In one particular example the pressure is from 40 to 60 psi.

Animal cells of the present invention include but are not limited to PER.C6®, HEK 293 (and variants), HER 911, A549, CHO, VERO, HELLA, MDCK, MRC-5, WI-38, Namwala, NSO, Chick embryos fibroblasts, primary mammalian cell lines (including Human), hybridomas, and insect cell lines.

Examples of suitable viruses that may be used to infect the animal cells include recombinant Adenovirus (all subgroups and serotypes and species in particular Ad-5, Ad-2, Ad-35, Ad-1, Ad-6 and adenovirus sub groups A, B, C, D, E, and F), Wild type Adenovirus (all serotypes and species), Chimeric constructs of adenovirus vectors (adenovirus/retrovirus, Ad/AAV and combination of all adenovirus serotypes e.g Ad-5/AD-2), rAAV (all sub groups and serotypes from all species in particular AAV2, AAV5, AAV1, AAV3, AAV4, AAV6), Chimeric constructs of AAV vectors (such as all combinations of AAV serotypes e.g AAV2/AAV5, AAV5/AAV1, AAV/Adenovirus), wt AAV (all serotypes from all species), Lenti virus, retrovirus (Mu-LV, X MuLv), HCV, HAV, HBV, WNV, SARS, EBOLA, EBV, Sendai, Vaccinia, PPV, CPV, Polio-1, Measles, yellow fever, Rubella, HIV, CMV, HPV, HSV. BVDV, SV-40, B-19, Alpha-virus, REO-3, Influenza, JEV (Japanese encephalitis) and Baculovirus.

In one embodiment of this invention animal cells are used for the production of viruses which are used in the manufacture of vaccines and viral vectors for gene therapy.

In a further embodiment the adenovirus used to infect the animal cells is a replication defective simian adenovirus. Typically these viruses contain an E1 deletion and can be grown on cell lines that are transformed with an E1 gene. They may also contain an E3 deletion in addition to the E1 deletion, in which case they can be grown on cell lines transformed with an E1 gene and an E3 gene. In a further aspect, they may contain an E4 deletion in addition to the E1, or E1 and E3 deletions. Examples of suitable Simian adenoviruses are viruses isolated from Chimpanzee. In particular C68 (also known as Pan 9) (See U.S. Pat. No 6,083,716) and Pan 5, 6 and Pan 7 (WO 03/046124) are suitable for use in the present invention. These viral vectors can be manipulated to insert a heterologous gene of the invention such that the gene product may be expressed. The use, formulation and manufacture of such recombinant adenoviral vectors is set forth in detail in WO 03/046142. Such replication defective adenoviruses can be produced on any suitable cell line in which the virus is capable of replication, for example HEK293 or PER.C6® cells. Preferred cell lines are complementing cell lines which provide the factors missing from the virus vector that result in its impaired replication characteristics, for example such cell lines may be transfected with the E1 gene, or with E1 and E3 genes. In a further aspect, they may also be transfected with the E4 gene.

The cells according to the invention may be present in the suspending fluid at a cell density of from 10³/ml to 10⁸/ml. In particular at a cell density of 10⁵/ml to 10⁶/ml. At lower cell density direct lysis from suspension of cell culture occurs, at higher cell density, direct lysis of static culture occurs. In order to vary the cell density of the suspension, the cells can be centrifuged down and resuspended in fluid. A concentrated cell suspension may also be derived from adherent cell cultures such as those in tissue culture flasks, roller bottles, or cell factories™ (Nunclon), or any variation thereof, whereby the cells are detached and the cell containing suspension centrifuged down and the resulting cell pellet resuspended in fluid.

A further method according to the invention comprises two or more conduit units bundled together in parallel to achieve higher throughput of cell suspension, and therefore increased efficiency of cell lysis. For example up to 100 conduit units may be bundled together. In particular the bundle may comprise from 10 to 100 conduit units, or from 10 to 50 conduit units.

The present invention also provides a method of recycling the suspension of cells. For example, by means of a circulating loop which exits and returns to the reservoir. In particular where the cell suspension passes through the restriction from 3 to 5 times, for example when it passes through 4 times. The optimum number of cycles may vary depending on the number of days post-infection of the cells.

In one embodiment, the invention provides a method of disrupting animal cells infected by virus in order to release viral particles which comprises:
a) a reservoir containing the culture fluid,
b) a circulating loop which exits and returns to the reservoir,
c) a pump to drive the culture fluid through the circulating loop, and
d) a restriction within the circulating loop adapted in use to cause lysis of the cells.

In one aspect of the invention the pressure applied to the cell suspension may change with each passage through the system, for example an increase in pressure with each passage. In another aspect of the invention, the cell density of the suspension may be varied, for example the cell density may be increased with each passage. The cell density may be varied, by way of example by removing the cell suspension from the reservoir after one passage, centrifuging the cell suspension and re-suspending the pellet in a reduced amount of suspending fluid. The re-suspended cell suspension can then be fed back into the reservoir for a further passage through the system.

The invention also provides an apparatus for the disruption of animal cells infected by virus in order to release viral particles which comprises:
a) a reservoir containing a suspension of the cells in a suspension liquid,
b) means to pressurise said suspension,
c) conduit,
d) communication means between said pressurisation means and said conduit to cause the pressurised suspension to flow through the conduit, and
e) means to receive output from the outlet end of the conduit.

The apparatus of the present invention may further be used to recover many types of intracellular products which are made using cultured cells. Examples of such intracellular products include: naturally occurring products such as proteins and polysaccharides, recombinant proteins including antibodies and enzymes, and viruses.

The invention further comprises a method of harvesting a virus grown in an animal cell comprising:
a) culturing animal cells either in anchorage mode or in suspension culture;
b) infecting the cultured cell at the required cell density with the virus;
c) after a suitable incubation period, passing the animal cells through the apparatus of the present invention as described above so that cells are disrupted and the virus is released; and
d) harvesting the released virus.

In one particular embodiment, cells infected with adenovirus, particularly Ad type 5 strain of adenovirus, are disrupted to harvest virus used to prepare a live viral vector for gene therapy.

Further aspects and features of the present invention are set forth in the exemplary embodiments of the invention which will now be described with reference to the accompanying Figures of drawings.

### BRIEF DESCRIPTION OF THE DRAWINGS

FIG. 1 is a schematic diagram of the capillary device in accordance with the present invention.

FIG. 2 is a schematic diagram of the capillary module device in accordance with the present invention.

FIG. 3 is a schematic diagram showing application of the invention to the recovery and purification of Adenovirus viral vector.

FIG. 4 is a schematic diagram detailing the cultivation of Adenovirus in an anchorage dependent cell line (HER 911).

FIG. 5 details the disruption of PGal-Adenovirus 5 infected PER.C6® cells using a commercially available homogeniser (Constant systems), under different chamber pressures and referenced to a freeze/thaw sample (FT).

FIGS. 6A-B show the disruption of HER 911 cells at various stages of culture age using the capillary device. Data shown from Tables 1 and 2.

FIGS. 7A-B show the disruption of HER 911 adenovirus infected cells after a one day (lys03/day01) and two day (lys03/day02) post infection period.

FIGS. 8A-B show the disruption of PER.C6® Adenovirus infected cells using the capillary lysis device.

FIG. 9 shows the effect of disruption pressure on release infectious titre using the capillary device.

FIG. 10 shows the effect of disruption pressure on Adenovirus infectious titre.

### DETAILED DESCRIPTION OF THE INVENTION

The disruption of animal cells can be achieved with a valve configuration using low pressure according to the invention. A typical pressure range required would be for example between 10-100 psi. This pressure is sufficient to both lyse the plasma and the nuclear membrane. Hence this will allow intracellular derived protein and nuclear derived material such as virus to be extracted. The low operating pressure results in a gentle disruption and avoids damaging the product.

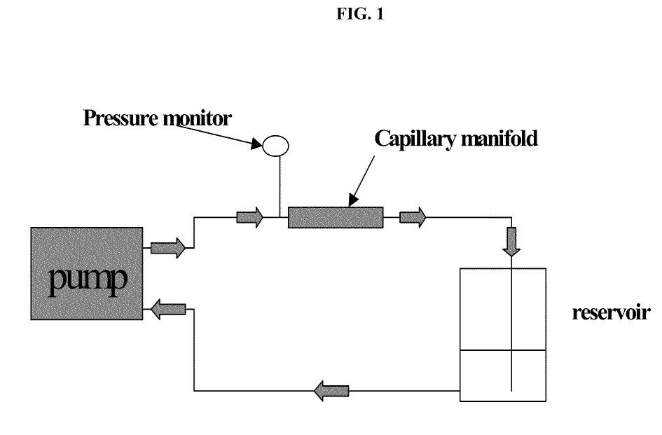

An alternative low-pressure method according to the invention has been developed employing a single channel capillary device as shown in FIG. 1.

Referring to FIG. 1, there is shown a peristaltic pump (1) of the present invention. The peristaltic pump (1) is used to drive the cell culture fluid (3) from a reservoir (2) through a capillary lysis device (5) which has a 0.13 mm capillary bore of 5 cm length. The exiting fluid is collected into the original reservoir (2). The material processed may then be further subjected to multiple cycles through the capillary device, at the same or higher pressure.

Alternatively, the exiting fluid is collected in a second reservoir (7) and then transferred back to the first reservoir (2) when the first reservoir is empty. This method of non continuous re-circulation has the effect of reducing the risk of product inactivation which could arise from continuous circulation.

Due to the capillary restriction a backpressure is generated when the culture fluid enters the capillary. This pressure can be regulated by the pump speed and is termed the “disruption pressure”.

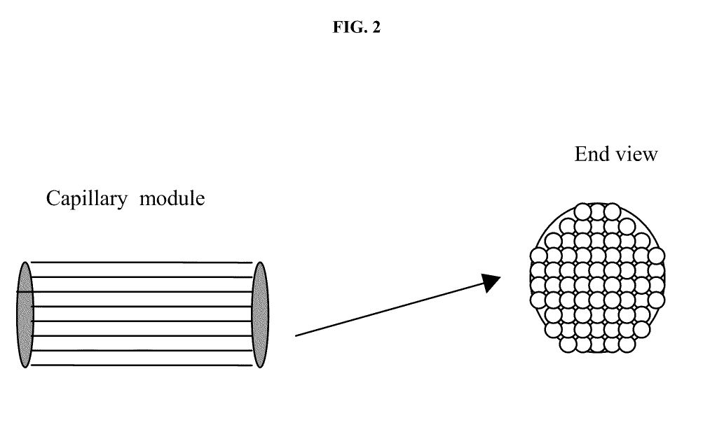

In the apparatus of the invention as shown in FIG. 1, a single capillary device was used. In a further embodiment of the invention the number of capillary elements in the capillary device can be increased which will increase the volumetric throughput. A diagram of such a design is shown in FIG. 2.

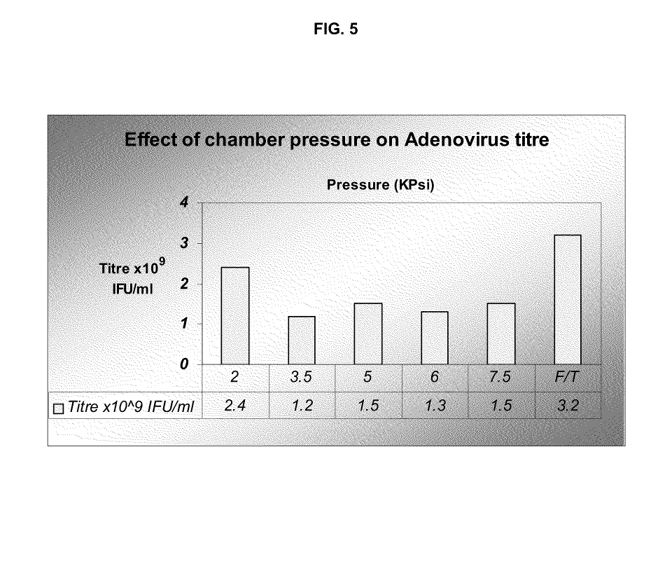

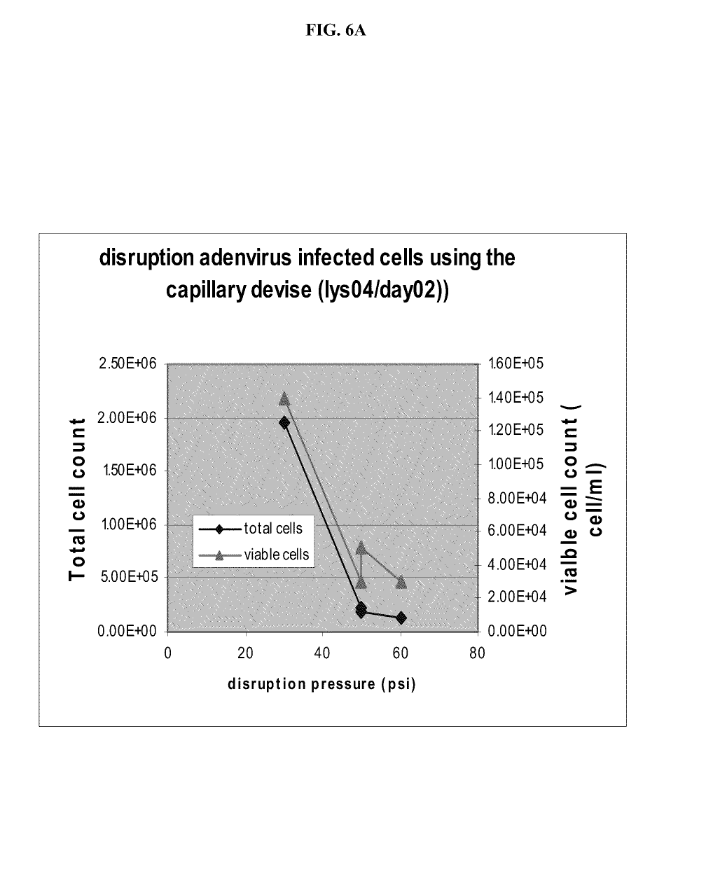

The capillary device can operate in a defined pressure range required for lysis of animal cells and recovery of intracellular material. Furthermore, the capillary device operates at lower pressures than current commercial mechanical cell disruption devices which operate at higher pressures (>1000 psi) (see for example FIG. 5). The current commercial mechanical devices also cause damage to shear sensitive products and the higher pressures used lead to generation of foam and possible product inactivation. Use of an apparatus as described in the present invention in a purification process will potentially reduce impurities as opposed to a detergent lysis step which may solubilise non-soluble impurities.

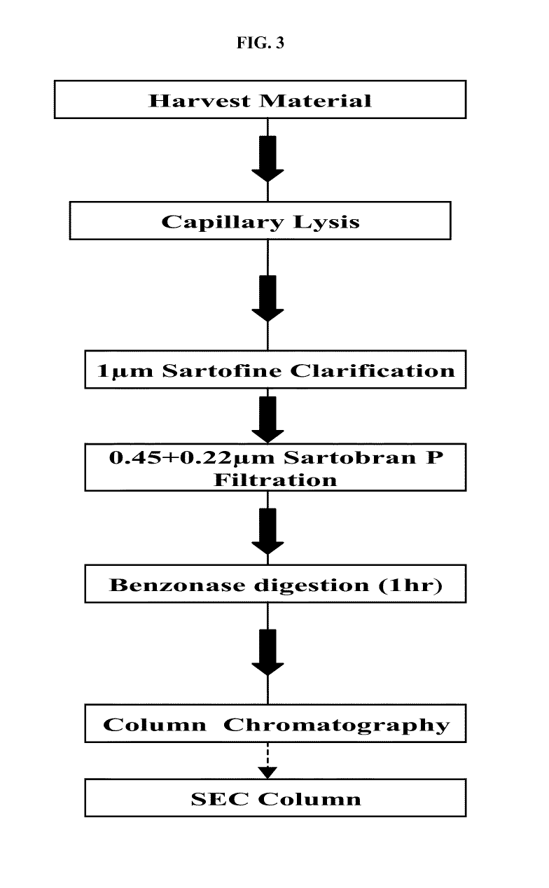

Thus an apparatus as described in the present invention could be utilised in the downstream processing and purification of any animal cell-derived viral product. In particular, applications involving viral vaccines or viral delivery vectors for gene therapy. More specifically to adenovirus and adeno-associated viral vectors. A typical adenovirus purification scheme using the capillary device of the present invention is shown in FIG. 3. Adenovirus may be cultured in either anchorage or suspension cell culture. Mammalian cells are grown to a defined cell density in suspension or percentage confluency if anchorage dependent cells are used using an appropriate culture media (such DMEM, MEM, Ham F-12) with or without the addition of serum. The cells are then infected with virus. Prior to this, the potency of virus seed is quantified and volume of virus used is calculated based on the number of virus particles required to infect a single cell (referred to as the Multiplicity of infection or MOI). An incubation period is allowed for the virus to infect the cells and hence generate progeny (500-10K). This incubation period is dependent on the cell line used, virus type, and the nature of the transgene. Based on prior knowledge of the process the virus is harvested in a defined time window post infection (this could be post infection 24 hrs-180 hr). The harvested cell suspension is then processed through the capillary devise, whereby virus infected cells are disrupted mechanically to release the adenovirus into the media. The disruptate pool is further clarified by using a depth filter (1.0 μm Sartofine) to remove insoluble debris. To minimise the risk of batch contamination a sterile 0.45 μm+0.22 μm filtration is undertaken.

Benzonase is added to the adenovirus suspension to reduce the nucleic acid content and to aid chromatographic resolution further downstream. The bulk solution is left to incubate at room temperature for 1 hour and then loaded onto an anion exchange chromatography column for further purification. Purified adenovirus is then eluted off the column in a defined salt gradient and can be further processed to attain an increase in purity and reduction in contaminants.

The following is intended as a non-limiting example of the invention. The specific embodiments described within the example may be modified as set forth in the claims.

### Example 1

### Experimental Evaluation of the Capillary Device

The following parameters were analysed with respect to the apparatus of the present invention.
disruption pressure and virus titre
optimal disruption pressure range
effect of harvest time on disruption pressure range

Cell counts (total and viable) were undertaken on the processed material and infectious adenovirus titre was determined by TCID50 assay.

Initial evaluation of the single channel capillary device was undertaken using both infected and non-infected cells. The cell lines HER 911 and PER.C6® were used both in suspension and adherent culture. A brief description of the cell cultivation is given below.

### Cultivation of Recombinant Adenovirus in Adherent HER 911 Cells

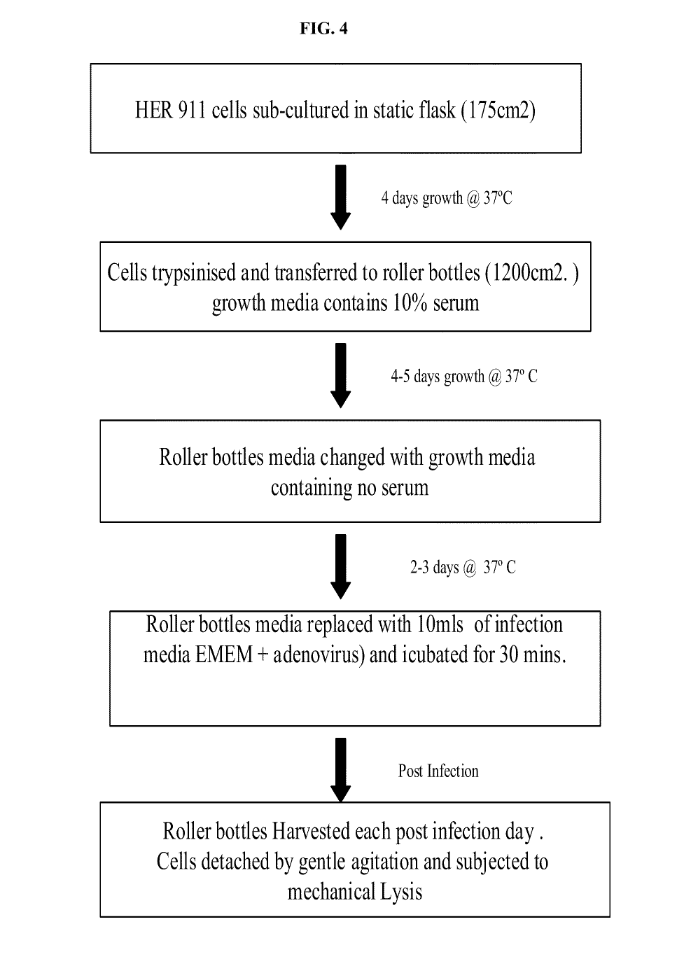

HER 911 were subcultured in static T flasks using growth media supplemented with 10% Fetal Bovine serum. On reaching confluency (approx. 4 days) the cells were further subcultured in roller bottles (1250 cm² surface area). A summary flow diagram is given in FIG. 4.

### Cultivation of Adenovirus in Suspension Cultures of PER.C6® Cell

Suspension cultures of PER.C6® were used to seed a 3 lt bioreactor containing 1 lt of media. The vessel was seeded at a cell density of 3×10⁵ cells/ml. Cells were cultured at 37 C and the cell density maintained by diluting the culture with fresh media. Once the desired cell density was reached the cells were infected with the adenovirus at a specified MOI and the temperature set point of the cultivation dropped to 34 C. The culture was harvested 4 days after infection.

Table 1 shows the systematic approach adapted to achieve cell disruption.

TABLE 1
routine processing of cells using the capillary device

| Cell culture | Capillary I.D | Initial disruption | |
|---|---|---|---|
| sample | and length | Pressure (psi) | Cycle number |
| Post infection | 0.13 mm/5 cm | 0 | 0 |
| day 2 | | | |
| Post infection | 0.13 mm/5 cm | 30 | 1 |
| day 2 | | | |
| Post infection | 0.13 mm/5 cm | 45-50 | 2 |
| day 2 | | | |
| Post infection | 0.13 mm/5 cm | 45-50 | 3 |
| day 2 | | | |
| Post infection | 0.13 mm/5 cm | 60 | 4 |
| day 2 | | | |

Total and viable cell counts were determined pre and post processing. Infectious Adenovirus titre was determined by TCID50.

### Results

### The Effect of Disruption Pressure on Cells

Comparative runs of lysis experiments all indicate that a common disruption mechanism is involved.

Cells harvested (post infection @ n days) from roller bottle culture (non-trypsinisation method) appear very clumped. Large clumped (cell sheet) material can be observed visually whilst smaller clumps are seen under a light microscope. The effect of the processing cycle 1 is de-clumping or disaggregation of cellular mass into smaller units or into unicellular material.

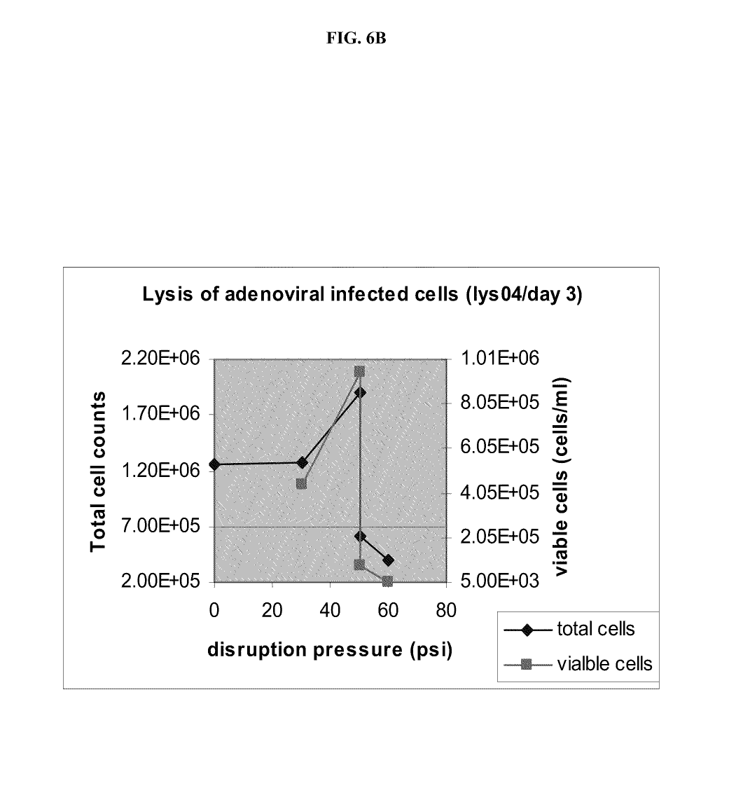

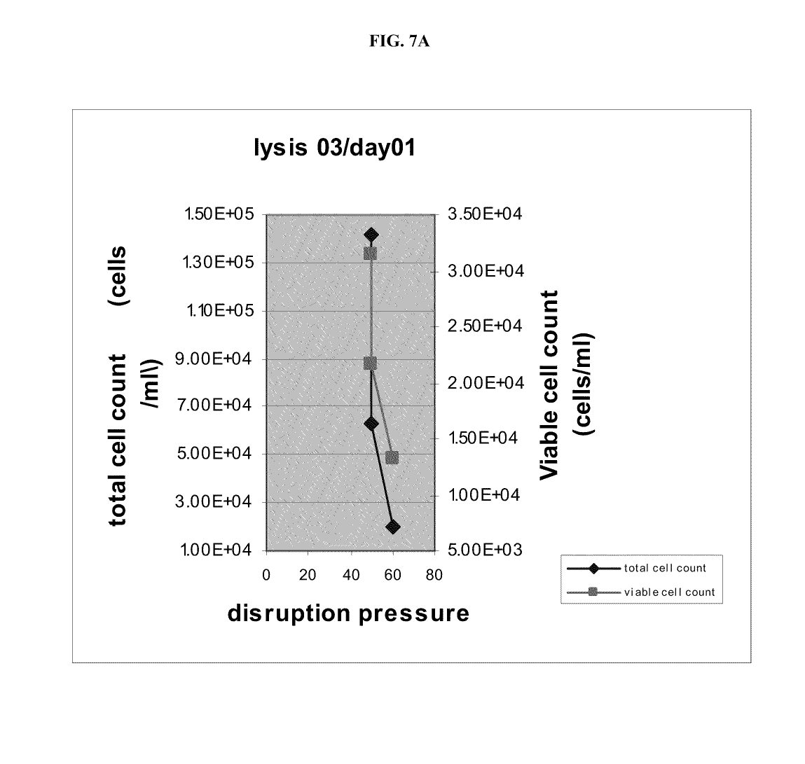

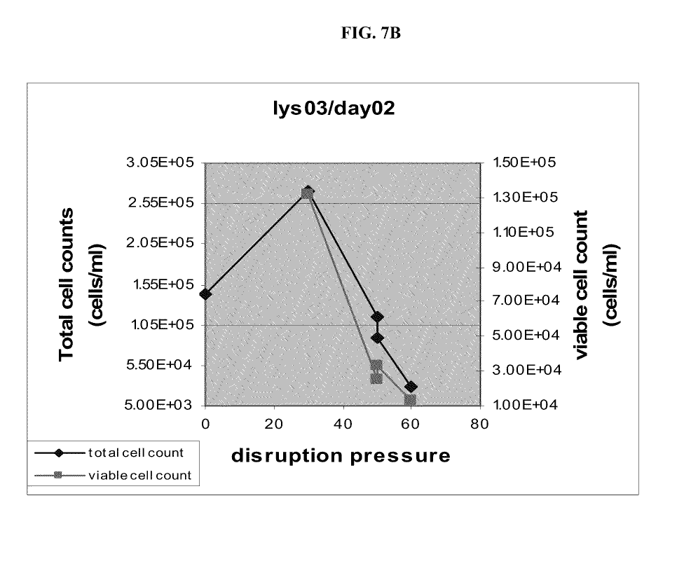

This is primarily achieved at the lower pressure range of 25-30 psi. The consequence of this is that a relative increase in cell count is observed (see FIGS. 5, 6 and 7 and table 2(a), 2(b)), possibly reflecting the true total cell count of the culture. Subjecting the same culture fluid sample to a higher disruption pressure range (45-50 psi) results in greater decline in total cell count and hence increase in cellular debris attributed to cellular attrition occurring in the capillary device. This is also reflected in the gradual decline in the viable cell counts indicating that cell lysis is occurring in the presence of the capillary device at this higher defined pressure range (45-50 psi). Increasing the disruption pressure to 60 psi results in a further decline in the total cell count (see FIG. 6).

TABLE 2(a)
Typical data generated using the capillary device for disruption
of PER.C6 ® cells infected with Recombinant Adenovirus
vector 37 hours post infection

| | Lysis | Total Cell | Non-Viable | | |
|---|---|---|---|---|---|
| Pass | Pressure | Count | Cell | % Cell | % Cell |
| No.: | (Psi)) | (Cell/Ml) | (Cells/Ml) | Viability | Reduction |
| 0 | 0 | 1.62E+06 | 5.85E+05 | 63.89 | 0.00 |
| 1 | 30 | 9.40E+05 | 4.30E+05 | 54.26 | 41.98 |
| 2 | 50 | 4.80E+05 | 4.20E+05 | 12.50 | 70.37 |
| 3 | 50 | 7.10E+05 | 6.00E+05 | 15.49 | 56.17 |
| 4 | 67.5 | 1.10E+05 | 1.10E+05 | 0.00 | 93.21 |

TABLE 2(b)
data generated using the capillary device for disruption
of PER.C6 ® cells infected with Recombinant Adenovirus
vector 58 hours post infection

| | Lysis | Total Cell | Non-Viable | | |
|---|---|---|---|---|---|
| | Pressure | Count | Cell | % Cell | % Cell |
| Pass | (Psi) | (Cell/Ml) | (Cells/Ml) | Viability | Reduction |
| 0 | 0 | 1.20E+06 | NA | | 0.00 |
| 1 | 30 | 1.28E+06 | 5.30E+05 | 41.41 | 95.58 |
| 2 | 50 | 7.50E+05 | 7.10E+05 | 55.47 | 94.08 |
| 3 | 50 | 3.50E+05 | 3.00E+05 | 23.44 | 97.50 |
| 4 | 65 | 1.40E+05 | 1.40E+05 | 10.94 | 98.83 |

### The Effect of Disruption Pressure on Infectious Virus Titre

Adenovirus usually propagates in the nucleus of cells. For effective release of the virion particles into the culture media disruption of the nuclear membrane and plasma membrane is required.

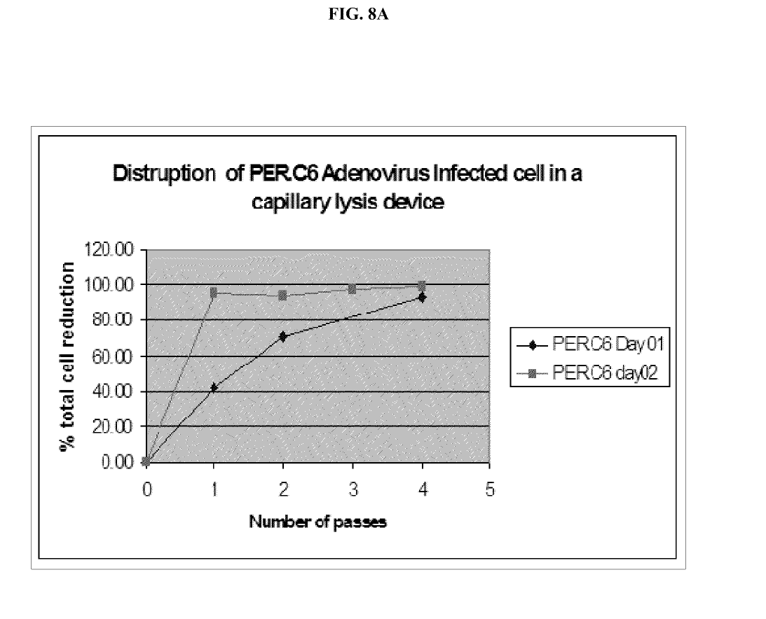

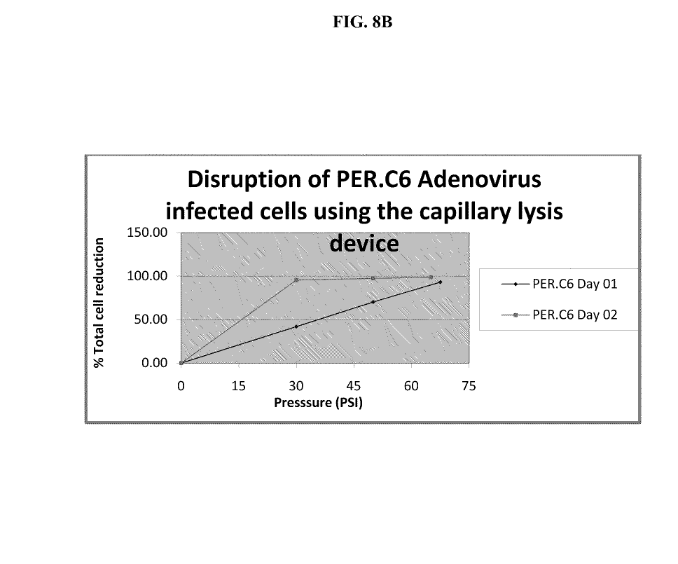

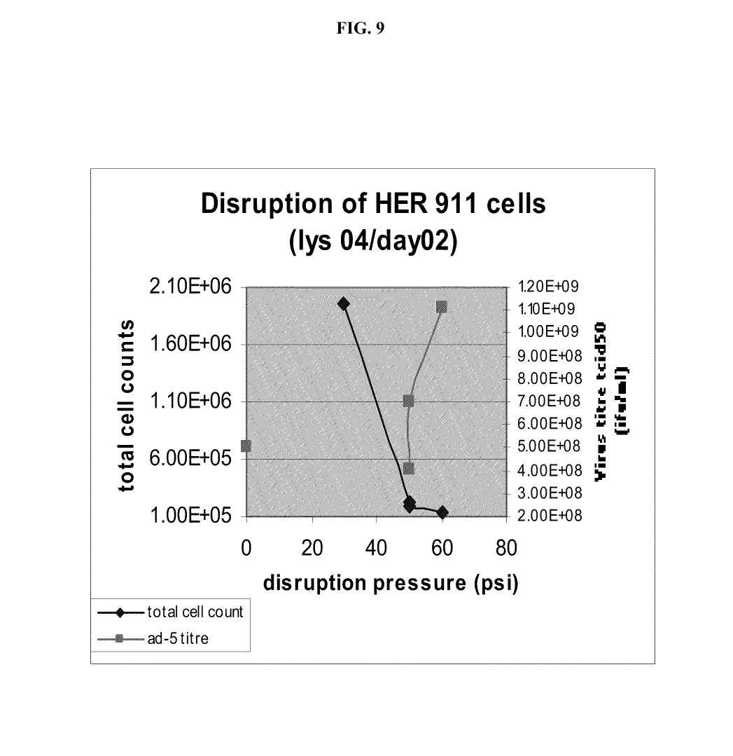

It can be observed that during the initial cycle (cycle 1 @ 30 psi) an increase in the infectious virus titre (see FIGS. 8, 9, and table 3) is observed relative to the supernant value (non-disrupted material). When the same sample is processed through the second cycle (cycle 2 @ 45-50 psi) a greater increase in the infectious titre is observed and on some occasions the peak titre is achieved in this second cycle.

TABLE 3
Effect of disruption pressure on HER911 adenovirus infected cells

| | Disruption | Cell total | Viable | | % cell | Tcid50 |
|---|---|---|---|---|---|---|
| Pass no.: | Pressure | Cell count | Cell/ml | % viability | Reduction | Titre |
| 0 | 0 | | | | | 5.03E+08 |
| 1 | 30 | 1.95E+06 | 1.40E+05 | 7.18 | 0 | |
| 3 | 50 | 1.90E+05 | 5.00E+04 | 26.32 | 90.3 | 7.01E+08 |
| 4 | 60 | 1.30E+05 | 3.00E+04 | 23.08 | 93.3 | 1.11E+09 |

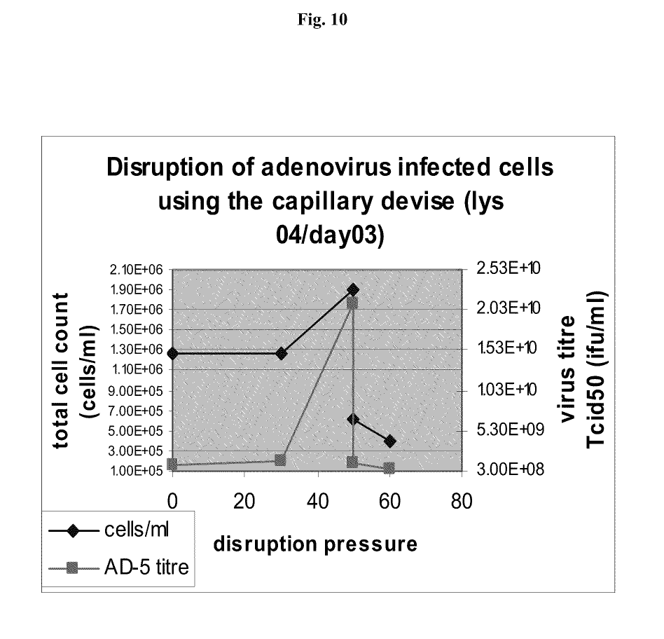

The above data indicates that a pressure range of 25-60 psi is sufficient for effective cell lysis and recovery of adenovirus. Furthermore excessive cycling of the disruptate through the capillary could result in viral inactivation as shown in FIG. 10, where a decline in infectious titre was observed. To prevent this, a defined time period and recycling rate is required to avoid product inactivation which could be due to excessive foaming or over-exposure to regions of high shear.

### Discussion

The data generated by the capillary device shows that effective lysis of HER 911 cells can be achieved between 40-60 psi. This is demonstrated in the rapid decline in total cell count (number of viable and non viable cells, see FIGS. 9, 10) and the corresponding decrease in cell viability indicating that cells are being continuously lysed in this pressure range. For effective recovery of adenovirus from HER 911 cells, the pressure range 25-60 psi is sufficient to disrupt virus infected cells at all stages of the post infection cycle. Hence the capillary device can be effectively applied at the pre-defined pressure range for the high yield recovery of adenovirus. Furthermore this capillary device can be used to recover other intracellular products from animal cells.

The mechanism for cell lysis in the capillary device is likely to be two-fold: At low pressure (i.e. low pump flow rates) the capillary device is highly effective in disaggregating the clumpy cell harvest. After the first cycle there is always an increase in the total cell count. This disaggregation is probably due to the clump size being too large to enter the capillary orifice. Hence loosely associated material is separated by shear forces, resulting in the generation of smaller clumps or unicellular material. As a consequence “ripened cells” with weakened cell membranes (i.e. those infected with virus) are disrupted more easily, this is reflected in the increase in virus titre. At higher disruption pressures (40-60 psi) the capillary starts to act as a disruption device. This is reflected in the rapid decline in the total cell count and generation of cellular debris (as analysed by the light microscope). A likely mechanism for this could be that the resulting increase in the pump flow causes a rise in the backpressure hence propelling the cell suspension from an area of relative low pressure (upstream of the pump) to a region of high pressure. This creates a high velocity through the capillary and propels the cells against the walls of the capillary causing the flow characteristic to change (laminar to turbulent flow). The accumulative effect of the cells impacting on the capillary walls together with friction and turbulence generated in the capillary will cause the plasma membrane and nuclear membrane to rupture. This can be observed in the decline of total and viable cell numbers and the presence of cellular debris due to attrition.

1-20. (canceled)

21. A method of extracting a virus from an animal cell infected with the virus, comprising the steps of
a) providing cells in a suspending fluid,
b) pressurising the suspension of cells to a pressure of from 5 psi to 1000 psi, and
c) causing the pressurised suspension to flow through an restriction having a maximum diameter of 0.5 mm at a flow rate so that the cell membrane is ruptured.

22. A method of extracting a virus from an animal cell infected with the virus, comprising the steps of
a) providing cells in a suspending fluid,
b) pressurising the suspension of cells, and
c) causing the pressurised suspension to flow through a conduit having a maximum diameter of 0.5 mm at a flow rate so that the cell membrane is ruptured.

23. A method according to claim 22 wherein the conduit has a bore size of from 0.05 mm to 0.2 mm.

24. A method according to claim 22 wherein the conduit is from 0.5 cm to 10 cm in length.

25. A method according to claim 24 wherein the conduit is from 4 cm to 6 cm in length.

26. A method according to claim 22 wherein two or more conduits are bundled together in parallel.

27. A method according to claim 21 wherein the restriction is a valve.

28. A method according to claim 21 wherein the suspension of cells is pressurised by means of a peristaltic pump.

29. A method according to claim 21 wherein the cell density in the suspension of cells is from 10³/ml to 10⁸/ml.

30. A method according to claim 21 wherein the pressure is from 10 to 250 psi.

31. A method according to claim 21 further comprising recycling of the suspension of cells.

32. A method according to claim 31 wherein the cell suspension passes through the restriction from 3 to 5 times.

33. An apparatus for the disruption of animal cells infected by virus in order to release viral particles which comprises:
a) a reservoir containing a suspension of the cells in a suspension liquid,
b) means to pressurise said suspension,
c) a conduit which is from 2 cm to 10 cm in length,
d) communication means between said pressurisation means and said conduit to cause the pressurised suspension to flow through the conduit, and
e) means to receive output from the outlet end of the conduit.

34. The apparatus of claim 33 wherein the conduit has a bore size of 0.05 mm-0.2 mm.

35. The apparatus according to claim 33 wherein two or more conduits are bundled together in parallel.

36. The apparatus of claim 33 wherein the suspension of cells is pressurised by means of a peristaltic pump.

37. The apparatus of claim 33 wherein the cell density in the suspending fluid is from 10³/ml to 10⁸/ml.

38. The apparatus of claim 33 wherein the pressure is from 10 to 250 psi.

39. The method of claim 21 wherein the animal cells are infected with adenovirus.

40. The apparatus of claim 33 wherein the animal cells are infected with adenovirus.
